(19) United States
(12) Patent Application Publication
(10) Pub. No.: US 2016/0178592 A1
(43) Pub. Date: Jun. 23, 2016

(54) APPARATUS FOR MEASURING CONCENTRATIONS OF DISSOLVED CONSTITUENTS

(71) Applicants: KURITA WATER INDUSTRIES LTD., Tokyo (JP); DKK-TOA CORPORATION, Tokyo (JP)

(72) Inventors: Shintarou MORI, Nakano-ku, Tokyo (JP); Yukimasa SHIMURA, Nakano-ku, Tokyo (JP); Tetsuya SATO, Shinjuku-ku, Tokyo (JP); Satoshi TAKIZAWA, Shinjuku-ku, Tokyo (JP)

(21) Appl. No.: 14/900,396

(22) PCT Filed: May 12, 2014
(86) PCT No.: PCT/JP2014/062576

§ 371 Date: Dec. 21, 2015

(30) Foreign Application Priority Data
Jun. 26, 2013 (JP) 2013-134047

(51) Int. Cl. G01N 33/18; G01N 21/25; G01N 21/59

(52) U.S. Cl. CPC G01N 33/18; G01N 21/59; G01N 21/255

(57) ABSTRACT

Provided is a device for measuring the concentration of a dissolved component, whereby the concentration of a plurality of types of dissolved components in water can be measured easily and with good precision. A device for measuring the concentration of a dissolved component has: a cell assembly 3 in which a plurality of cells 1 are connected by a joint member 2 and integrated, and a measurement reagent is accommodated in each of the cells 1; and a measuring main body 5 having a measurement unit (light-emitting element 8 and light-receiving element 9) for measuring transmittance and absorbance, the cells 1 of the cell assembly 3 being inserted into the measurement unit. A cap 4 is detachably mounted to each cell 1.

### FIELD OF THE INVENTION

The present invention relates to an apparatus for measuring the concentrations of plural types of dissolved constituents in water contained in a cooling water system, boiler water, or the like.

In order to operate a water-using plant in a safe and efficient manner, it is necessary to control water quality at a level suitable for the plant. Accordingly, water quality analysis plays an important role. For example, silica may adhere in the form of silica scale due to concentration performed in an open-circulating cooling water system. In particular, if the adhesion of scale to a heat exchanger is progressed, the thermal efficiency of the heat exchanger may be reduced and ease of passing water through the heat exchanger may be reduced. If no action is taken, this may cause serious defects such as closedown to occur.

Phosphate, which serves as an anticorrosive in a heat exchanger, pipes, and the like, is controlled to remain in water at a certain concentration. If the concentration of phosphate runs short, corrosion may occur and progress and, as a result, perforation may occur, which results in defects such as the closedown of the plant.

Acid consumption (pH: 4.8) is decomposed inside a boiler due to heat and pressure, and the resulting carbonic acid gas may cause steam condensing pipes to be corroded. If these pipes are corroded, the amounts of water and thermal energy recovered may be reduced, which reduces the operating efficiency.

In order to prevent the above-described defects from occurring, it is required to periodically analyze water brought from a field. Specifically, in accordance with JIS K0101, the silica concentration in the water is measured by a molybdenum-yellow absorptiometric method, phosphate is measured by a molybdenum-blue absorptiometric method, and acid consumption (pH: 4.8) is measured by a titrimetric method. However, these analyses require large amounts of time and manpower since the water is manually analyzed after being brought from a field.

An anionic polymer such as an acrylic acid polymer, an acrylic acid copolymer, a maleic acid polymer, or a maleic acid copolymer is added to a cooling-water system and a water system including steam-generating equipment such as a boiler for the purpose of water treatment, such as prevention of scale, corrosion, and contamination. While the anionic polymer serves as a high-performance scale inhibitor, it is essential to manage the concentration of the anionic polymer in a targeted water system in order to make full use of the scale-inhibition capability of the anionic polymer. A method for measuring the concentration of such an anionic polymer in water is described in Patent Literature 1, in which a reagent is added to test water taken from a water system and reacted with an anionic polymer such that the test water is turned cloudy, and the test water is subjected to a measurement using a portable measurement apparatus capable of emitting visible light having a wavelength of 400 to 900 nm in order to conduct a turbidimetric analysis (claim 1 of Patent Literature 1). In Patent Literature 1, the portable measurement apparatus includes an optical measurement unit including a measurement cell provided with a lid, an opening portion to which the measurement cell is attachable, a visible-light-emitting portion disposed on a side surface of the opening portion, and a light-receiving portion that receives the resulting transmitted or reflected light; a light-shielding cap that covers the optical measurement unit in order to shield the optical measurement unit from light; a data processing unit that receives an electrical signal sent from the optical measurement unit and perform a computation; and a display unit that displays the results obtained in the data processing unit (claim 5 of Patent Literature 1).

### LIST OF LITERATURE

### Patent Literature

Patent Literature 1: Japanese Patent Publication 2006-38462 A

### Object and Summary of the Invention

### Object of the Invention

Measuring the concentrations of dissolved constituents by an absorptiometric method, a titrimetric method, and the like requires considerably large amounts of time and manpower. The method for measuring an anionic polymer which is disclosed in Patent Literature 1 also requires large amounts of time and manpower because it is necessary to take plural reagents and test water in exact amounts in order to achieve an accurate measurement.

An object of the present invention is to provide an apparatus for measuring the concentrations of dissolved constituents with which the concentrations of plural types of dissolved constituents in water can be easily measured with accuracy.

### SUMMARY OF THE INVENTION

The apparatus for measuring concentrations of dissolved constituents of the invention includes a cell assembly including a plurality of cells and a joint member, the cells being joined to one another with the joint member thereby being integrated into a single piece, and the cells each containing a measurement reagent; and a measurement device main body including a measuring unit for measuring transmittance or absorbance into which the cells of the cell assembly are to be inserted.

In the apparatus for measuring concentrations of dissolved constituents of the present invention, it is preferable that the cells are each provided with a cap that is detachable or reclosable.

In the apparatus for measuring concentrations of dissolved constituents of the present invention, it is preferable that the cells of the cell assembly are arranged in a line at regular intervals, and openings into which the respective cells are to be inserted are formed in an upper surface of the measurement device main body at the same intervals as those of the cell assembly.

In the apparatus for measuring concentrations of dissolved constituents of the present invention, it is preferable that the apparatus further includes a light-shielding lid that covers an upper portion of the cell assembly placed on the measurement device main body.

### Advantageous Effects of Invention

In the present invention, plural measurement cells each contain an aqueous solution of a different type of measurement reagent. Therefore, the number of operations in which the cells are charged with a liquid in a measurement field is only two; an operation in which test water is charged into the cells and an operation in which a second reagent is charged into the cells as needed. Thus, the concentrations of plural types of dissolved constituents can be easily measured. In addition, the measurement cells can be easily handled since the measurement cells are joined to one another and thereby integrated into a single piece.

### BRIEF DESCRIPTION OF DRAWINGS

FIG. 1 is a schematic perspective view of an apparatus for measuring the concentrations of dissolved constituents according to an embodiment.

FIG. 2 is a cross-sectional view of the apparatus illustrated in FIG. 1 which is taken along the line II-II.

### DESCRIPTION OF EMBODIMENTS

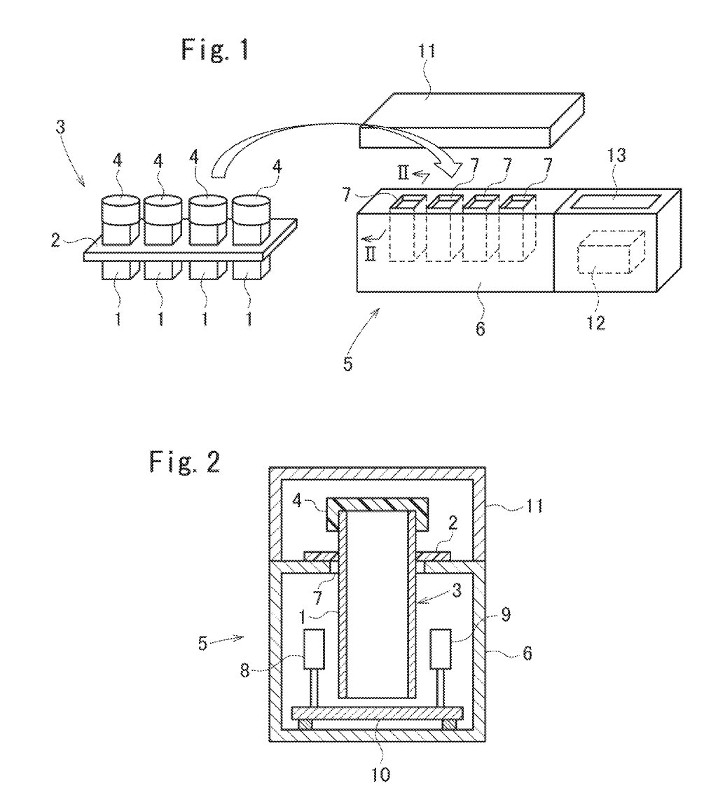

An embodiment is described below with reference to FIGS. 1 and 2.

As illustrated in FIG. 1, the apparatus for measuring the concentrations of dissolved constituents includes a cell assembly 3 including plural rectangular prismatic cells 1 and a joint member (in this embodiment, joint plate) 2. The cells 1 are joined to one another with the joint member 2 and thereby integrated into a single piece. The cells 1 are arranged in a line at regular intervals. In this embodiment, the joint member 2 is composed of a resin. Rectangle holes are formed in the plate 2, and the cells 1 are fitted into the respective holes.

The cells 1 each contain a different type of reagent in a predetermined amount in order to analyze a different dissolved constituent. The cells 1 are each hermetically sealed with a cap 4 detachably attached to the cell 1.

Plural openings 7 are formed in the upper surface of a bodyshell 6 included in a measurement device main body 5 at the same intervals as those at which the cells 1 are arranged. The cells 1 of the cell assembly 3 are inserted into the respective openings 7.

The bodyshell 6 houses light-emitting elements 8 disposed in front of the respective cells 1 inserted through the openings 7 and light-receiving elements 9 disposed across the respective cells 1 from the light-emitting elements 8. The elements 8 and 9 are disposed on a common circuit board 10. The light-emitting elements 8 and the light-receiving elements 9 each measure light transmittance or absorbance at a wavelength corresponding to the color in which the solution contained in a corresponding one of the cells 1 appears.

A light-shielding lid 11 is detachably attached to the upper surface of the bodyshell 6 so as to cover the upper portion of the cell assembly 3 inserted in the bodyshell 6.

Although not illustrated, an anchoring protrusion with which the light-shielding lid 11 is anchored to the bodyshell 6 is formed on the upper surface of the bodyshell 6.

The bodyshell 6 also houses a circuit unit 12 including, for example, a driving circuit used for operating the light-emitting elements 8 and the light-receiving elements 9 and a microcomputer that computes transmittances or absorbances in accordance with the amounts of light received by the respective light-receiving elements 9 and the concentrations of dissolved constituents by substituting the transmittances or absorbances into a predetermined calibration expression. The computed results are displayed on a display unit 13 composed of liquid crystal or the like, which is disposed on the outer surface of the bodyshell 6.

The concentrations of dissolved constituents in water can be measured using the apparatus for measuring the concentrations of dissolved constituents in the following manner. The caps 4 of the cells 1 are removed. Sampled test water is charged into each of the cells 1 in a predetermined amount. As needed, a second reagent is added to the cells 1. Subsequently, the caps 4 are attached to the cells 1, and the cell assembly 3 is shaken in order to mix the test water with the reagent contained in each cell 1. The resulting cell assembly 3 is positioned above the bodyshell 6 and subsequently brought down such that the lower portions of the cells 1 are inserted inside the bodyshell 6 through the respective openings 7 and the joint plate 2 is placed on the upper surface of the bodyshell 6. Thus, the lower portions of the cells 1 are each interposed between a corresponding pair of the light-emitting element 8 and the light-receiving element 9. After the bodyshell 6 is covered with the light-shielding lid 11, a switch (not illustrated) disposed on the bodyshell 6 is turned on. Upon turning the switch on, a control circuit included in the circuit unit 12 outputs a control signal, which causes the pairs of the light-emitting elements 8 and the light-receiving elements 9 to operate sequentially. Subsequently, the transmittance or absorbance of the liquid contained in each cell 1 is determined, and the concentrations of dissolved constituents in the test water are computed. The computed results are displayed on the display unit 13.

In this manner, the concentrations of plural types of dissolved constituents in the test water can be easily measured at once.

When absorbance or transmittance is measured by operating the light-emitting elements 8 and the light-receiving elements 9, each pair of the elements 8 and 9 may be operated at any timing during the period while color after the reaction is stable. Alternatively, the transmittances or absorbances of the cells may be measured sequentially without a pause. In another case, the transmittances or absorbances of the cells may be measured by staggering the measurement time in consideration of the reaction times suitable for measuring the dissolved constituents. In order to minimize the interference of measurements, it is preferable to measure absorbance or transmittance by staggering the time at which light is emitted.

In order to equalize the amounts of test water to be charged into the cells, the concentrations of the reagents are adjusted such that the concentration range in which each item is to be measured is achieved. This makes it possible to quickly measure and charge the test water into the cells without the risk of mismeasuring the amount of test water.

The caps 4 of the cells 1 are preferably one-touch caps in order to increase ease of operation in the analysis in a field. The directions in which the caps 4 are opened and closed are preferably set to be the same in order to increase ease of opening and closing the caps.

The dissolved constituents measured using the apparatus according to the present invention are constituents that can be measured using light transmittance or absorbance. Examples of such constituents include pH, silica, chloride ion, sulphate ion, Ca, Mg, acid consumption (pH: 4.8), acid consumption (pH: 8.3), phosphate ion, polymer, chlorine, sulfite ion, hydrazine, nitrate, nitrite, and ferrous ion. These constituents can be measured using, for example, the reagents at the measurement wavelength as described in Table 1.

TABLE 1

| | Measurement | | |
|---|---|---|---|
| Measurement target | wavelength (nm) | Reagent 1 | Reagent 2 |
| Silica | 420 | Hydrochloric acid: 0.3 vol % + ammonium molybdate: 1 wt % | |
| Acid consumption (pH: 4.8) | 620 | Succinic acid: 0.2 wt %-bromocresol green: 0.01 wt % | |
| Phosphate ion | 660 | Sulfuric acid: 1 vol %, ammonium molybdate: 0.1 wt % | Ascorbic acid: 100% (powder) |
| Anionic polymer | 528 | EDTA: 0.7 wt % | Benzethonium chloride: 5 wt % |
| Calcium ion | 570 | OCPC: 0.004 wt %, diethanolamine: 0.03 vol %, diethylamine: 0.03 | |
| | | vol %, polyvinyl pyrrolidone: 0.2 wt %, hydroxyquinoline: 0.3 wt % | |
| Magnesium ion | 520 | Xylidyl blue: 0.05 wt %, triethanolamine: 0.1 wt %, thorium | |
| | | sulfate: 0.2 wt %, GEDTA: 0.01 wt %, tetraethylenepentaamine: | |
| | | 0.1 wt %, disodium hydrogen phosphate: 0.2 wt % | |
| Ferrous ion | 510 | Phenanthroline: 0.1 wt %, sodium acetate: 7 wt % | |

There is no limitation to reagents charged into the cells in advance in order to detect whether the amounts of reagents and test water are suitable for measuring dissolved constituents. Charging the cells with the materials described in Table 2, which reacts with fluorescence or light having a wavelength different from the wavelength to be measured, makes it possible to confirm whether the reagent is suitable for measuring the dissolved constituent or whether the amount of test water charged is suitable for measuring the dissolved constituent.

TABLE 2

| | Excitation | Emission |
|---|---|---|
| | wavelength | wavelength |
| Name | (maximal) | (maximal) |
| Fluorescein | 495 | 519 |
| Tetramethylrhodamine | 552 | 578 |
| Texas Red | 595 | 613 |

Although the present invention has been described in detail with reference to a particular embodiment, it is apparent to a person skilled in the art that various modifications can be made therein without departing from the spirit and scope of the present invention.

The present application is based on Japanese Patent Application No. 2013-134047 filed on Jun. 26, 2013, which is incorporated herein by reference in its entirety.

1. An apparatus for measuring concentrations of dissolved constituents, comprising:
a cell assembly including a plurality of cells and a joint member, the cells being joined to one another with the joint member thereby being integrated into a single piece, and the cells each containing a measurement reagent; and
a measurement device main body including a measuring unit for measuring transmittance or absorbance into which the cells of the cell assembly are to be inserted,
the cells each containing a different type of reagent in a predetermined amount in order to analyze a different dissolved constituent.

2. The apparatus for measuring concentrations of dissolved constituents according to claim 1, wherein the cells are each provided with a cap that is detachable or reclosable.

3. The apparatus for measuring concentrations of dissolved constituents according to claim 1,
wherein the cells of the cell assembly are arranged in a line at regular intervals, and
wherein openings into which the respective cells are to be inserted are formed in an upper surface of the measurement device main body at the same intervals as those of the cell assembly.

4. The apparatus for measuring concentrations of dissolved constituents according to claim 3, the apparatus further comprising a light-shielding lid that covers an upper portion of the cell assembly placed on the measurement device main body.

5. The apparatus for measuring concentrations of dissolved constituents according to claim 3, the apparatus further comprising generation elements and light-receiving elements, each light-emitting element and light-receiving element being disposed across a corresponding one of the cells; and a controller for operating pairs of the light-emitting elements and the light-receiving elements sequentially.
